(19) United States
(12) Patent Application Publication
(10) Pub. No.: US 2010/0021982 A1
(43) Pub. Date: Jan. 28, 2010

(54) LIGHT-SENSITIVE CONSTRUCTS FOR INDUCING CELL DEATH AND CELL SIGNALING

(76) Inventor: Stefan Herlitze, Cleveland Heights, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/518,187

(22) PCT Filed: Dec. 6, 2007
(86) PCT No.: PCT/US07/86617

§ 371 Date: Jun. 8, 2009

Related U.S. Application Data
(60) Provisional application No. 60/873,188, filed on Dec. 6, 2006.

(51) Int. Cl. C12N 13/00

(52) U.S. Cl. 435/173.1

(57) ABSTRACT

A light-sensitive G-protein coupled receptor includes a light sensitive extracellular domain and a hetorologous intracellular domain capable of modulating an intracellular signaling pathway.

### RELATED APPLICATION

This application claims priority from U.S. Provisional Application No. 60/873,188, filed Dec. 6, 2006, the subject matter, which is incorporated herein by reference.

### GOVERNMENT FUNDING

This invention was made with government support under Grant No. NIH NS047752 awarded by the National Institutes of Health. The United States government has certain rights in the invention.

### FIELD OF THE INVENTION

The present invention relates to a light-sensitive constructs or probes for inducing cell death or signaling and light-sensitive constructs or probes for treating skin disorders and altering the cellular state of epithelial cells.

### BACKGROUND

G-protein coupled receptors (GPCRs) constitute a major class of proteins responsible for transducing a signal within a cell. GPCRs have three structural domains: an amino terminal extracellular domain, a seven transmembrane domain containing seven transmembrane domains, three extracellular loops, and three intracellular loops, and a carboxy terminal intracellular domain. Upon binding of a ligand to an extracellular portion of a GPCR, a signal is transduced within the cell that results in a change in a biological or physiological property of the cell. GPCRs, along with G-proteins and effectors (intracellular enzymes and channels modulated by G-proteins), are the components of a modular signaling system that connects the state of intracellular second messengers to extracellular inputs.

The GPCR protein superfamily can be divided into five families: Family I, receptors typified by rhodopsin and the β-2-adrenergic receptor and currently represented by over 200 unique members (Dohlman et al., Annu. Rev. Biochem. 60:653-688 (1991); Family II, the parathyroid hormone/calcitonin/secretin receptor family (Juppner et al., Science 254:1024-1026 (1991); Lin et al., Science 254:1022-1024 (1991); Family III, the metabotropic glutamate receptor family (Nakanishi, Science 258 597:603 (1992)); Family IV, the cAMP receptor family, important in the chemotaxis and development of D. discoideum (Klein et al., Science 241:1467-1472 (1988)); and Family V, the fungal mating pheromone receptors such as STE2 (Kurjan, Annu. Rev. Biochem. 61:1097-1129 (1992)).

There are also a small number of other proteins which present seven putative hydrophobic segments and appear to be unrelated to GPCRs; they have not been shown to couple to G-proteins. Drosophila expresses a photoreceptor-specific protein, bride of sevenless (boss), a seven-transmembrane-segment protein which has been extensively studied and does not show evidence of being a GPCR (Hart et al., Proc. Natl. Acad. Sci. USA 90:5047-5051 (1993). The gene frizzled (fz) in Drosophila is also thought to be a protein with seven transmembrane domains. Like boss, fz has not been shown to couple to G-proteins (Vinson et al., Nature 338:263-264 (1989).

G proteins represent a family of heterotrimeric proteins composed of α, β, and γ subunits, that bind guanine nucleotides. These proteins are usually linked to cell surface receptors, e.g., receptors containing seven transmembrane domains. Following ligand binding to the GPCR, a conformational change is transmitted to the G protein, which causes the α-subunit to exchange a bound GDP molecule for a GTP molecule and to dissociate from the β-γ-subunits. The GTP-bound form of the .alpha.-subunit typically functions as an effector-modulating moiety, leading to the production of second messengers, such as cAMP (e.g., by activation of adenyl cyclase), diacylglycerol or inositol phosphates. Greater than 20 different types of α subunits are known in humans. These subunits associate with a smaller pool of β and γ subunits. Examples of mammalian G proteins include Gi, Go, Gq, Gs and Gt. G proteins are described extensively in Lodish et al., Molecular Cell Biology, (Scientific American Books Inc., New York, N.Y., 1995), the contents of which are incorporated herein by reference. GPCRs, G proteins and G protein-linked effector and second messenger systems have been reviewed in The G-Protein Linked Receptor Fact Book, Watson et al., eds., Academic Press (1994).

### SUMMARY OF THE INVENTION

The present invention relates to a method of modulating cell signaling in neoplastic cells. In the method, light-sensitive transmembrane proteins are expressed from the neoplastic cells. The light sensitive transmembrane proteins modulate polarization of the cells upon exposure of the cells to a wavelength of light. The neoplastic cells expressing the light sensitive transmembrane proteins are exposed to the wavelength of light.

In an aspect of the invention, the modulation of cell signaling and/or the modulation of polarization of the cell can induce cell apoptosis or death. The light-sensitive transmembrane protein can modulate ion transport or fluxes across the cell membrane upon exposure to light.

In another aspect of the invention, the neoplastic cells can comprise at least one of a tumor of skin or a tumor of epithelial cells. The light-sensitive transmembrane protein can comprise at least one of channelrhodopsin, bacteriorhodopsin, vertebrate rhodopsin, invertebrate rhodopsin, or light activatable adenylate cyclase. The channelrhodopsin can include at least one of ChR1, ChR2, or ChR3.

The present invention also relates to a method of inducing cell death in neoplastic cells. In the method, light-sensitive transmembrane proteins are expressed from the neoplastic cells. The light sensitive transmembrane proteins modulate polarization of the cells upon exposure of the cells to a wavelength of light. The neoplastic cells expressing the light sensitive transmembrane proteins are exposed to the wavelength of light. The light-sensitive transmembrane protein can comprise at least one of channelrhodopsin, bacteriorhodopsin, vertebrate rhodopsin, invertebrate rhodopsin, or light activatable adenylate cyclase. The channelrhodopsin can include at least one of ChR1, ChR2, or ChR3.

The present invention further relates to a neoplastic cell comprising a light-sensitive transmembrane protein. The light sensitive transmembrane proteins modulates polarization of the cell upon exposure to a wavelength of light. The light-sensitive transmembrane protein can comprise at least one of channelrhodopsin, bacteriorhodopsin, vertebrate rhodopsin, invertebrate rhodopsin, or light activatable adenylate cyclase. The channelrhodopsin can include at least one of ChR1, ChR2, or ChR3.

### BRIEF DESCRIPTION OF THE DRAWINGS

FIG. 1 illustrates plots showing vertebrate rhodopsin modulates GIRK and P/Q-type Ca²⁺ channels via Gi/o-PTX-sensitive pathways. (A) K⁺ current traces of GIRK1/2 channels coexpressed with RO4 or mAChR-M2 in HEK293 cells before, during, and after light stimulation (Left) or 10 μM Carb application (Right). Currents were elicited by 500-ms voltage ramps from −100 to +50 mV. (B) Comparison of the GPCR-induced current increase in the presence and absence of 5 nmol PTX. (C) Time course traces of GPCR-mediated activation of GIRK currents. GIRK currents were recorded at −60 mV. (D) Comparison of the time constants of the GPCR-induced GIRK current changes before and after GPCR activation. (E) Ba²⁺ current traces of P/Q-type Ca²⁺ channels (α₁2.1, β_{1b}, and α_{2δ} subunits) coexpressed with RO4 or mAChR-M2 in HEK293 cells before, during, and after light stimulation (Left) or 10 μM Carb application (Right). (F) GPCR-induced depolarizing shift in the voltage dependence of activation curve of P/Q-type Ca²⁺ currents. Currents were elicited from a holding potential of −60 mV by 5-ms-long, 5-mV voltage steps from −10 to+65 mV. Relative tail currents were plotted against the voltage pulses. (G) Time course traces of GPCR-mediated inhibition of P/Q-type Ca²⁺ currents. Ba²⁺ currents were elicited by voltage pulses from −60 to +20 mV and measured every s. (H) Comparison of the time constants of the GPCR-induced P/Q-type channel current changes before and after GPCR activation. Throughout all experiments number in parentheses indicate the number of experiments and statistical significance as indicated (*, P<0.05; **, P<0.01, ANOVA).

FIG. 2 illustrates functional expression and characterization of vertebrate rhodopsin in cultured hippocampal neurons. (A) Colocalization of RO4 and synaptobrevin in cultured hippocampal neurons. (Left) Fluorescence patterns of neurons from low-density hippocampal cultures transfected with RO4 reveal a punctate staining. RO4 was detected with an anti-RO4 antibody and visualized with an Alexa 488-coupled secondary antibody. (Center) Hippocampal cells were stained with an antisynaptobrevin II antibody and visualized with an Alexa 568-coupled secondary antibody. (Right) Overlay of RO4 and synaptobrevin II staining. Yellow indicates colocalization. (B) RO4 induced voltage change during a long (Upper) and short (Lower) light pulse. (C) Average GPCR (RO4, GABAB)-induced hyperpolarization of cultured hippocampal neurons. Throughout the experiments GABAB receptors were activated by application of 50 μM baclofen (Bacl). (D) Time course of GPCR (RO4, GABAB)-induced hyperpolarization and recovery from hyperpolarization after switching off the light or washing out baclofen. (E) Voltage traces of current-induced (30 pA) neuronal firing of cultured hippocampal neurons before and during light activation of RO4. (F) Comparison of the number of action potentials measured after current injection for a neuron before and during light activation of RO4. (G) Comparison of EPSC amplitude before, during, and after light application for EPSCs measured in autaptic hippocampal cultures expressing RO4. EPSCs in autaptic hippocampal neurons were elicited by 2-ms voltage pulses from −60 to +10 mV. (H) Comparison of GPCR (RO4, GABA_{B})-induced EPSC inhibition measured in autaptic hippocampal neurons. (I) Time constants of GPCR (RO4, GABA_{B})-induced EPSC inhibition and release from inhibition. EPSCs were elicited every 5 s as described in G. (J) Autaptic EPSC traces elicited by 2-ms voltage pulses from-60 to +10 mV separated by 50 ms (20-Hz stimulation) before and after light activation of RO4. (K) Comparison of paired pulse facilitation before and after GPCR (RO4, GABA_{B}) activation for a 20-Hz stimulation protocol. The amplitude of the second EPSC was compared with the first EPSC.

FIG. 3 illustrates functional expression and characterization of green algae ChR2 in cultured hippocampal neurons. (A) Colocalization of ChR2 and synaptobrevin in cultured hippocampal neurons. (Left) Fluorescence patterns of neurons from low-density hippocampal cultures transfected with GFP-ChR2 reveal a punctate staining. (Center) Hippocampal cells were stained with an antisynaptobrevin II antibody and visualized with an Alexa 568-coupled secondary antibody. (Right) Overlay of GFP-ChR2 and synaptobrevin II staining. Yellow indicates colocalization. (B) Voltage traces of ChR2-induced neuronal firing of cultured hippocampal neurons for light stimuli with increasing duration. (C) Voltage traces of ChR2-induced neuronal firing of cultured hippocampal neurons for light stimuli with different frequencies. (D) Number of action potentials measured in neurons expressing ChR2. Action potentials were elicited by a train of 10 stimuli for different light stimulation frequencies with a light duration of 5 ms. (E) Light activation of ChR2 expressed in excitatory (Upper) or inhibitory (Lower) presynaptic neurons induce activation or inhibition in the paired postsynaptic neurons. (E1 and E4) EPSC (Upper) or IPSC (Lower) were elicited by a 2-ms voltage pulse from −60 to +10 mV in the postsynaptic autaptic neuron. (E2 and E5) Light activation of the excitatory and inhibitory presynaptic cells expressing ChR2 induced EPSC (Upper) or IPSC (Lower) on the postsynaptic, autaptic neurons. (E3) Presynaptically (excitatory) light induced spiking or subthreshold depolarization (Inset) of the postsynaptic neuron after a single 5-ms light pulse (Left) or a 10-Hz/5-ms light stimulation protocol (Right). Five light pulses were applied. (E6) Presynaptically (inhibitory) light induced hyperpolarization of the postsynaptic neurons after a single 5-ms light pulse. (E7) Schematic diagram of the neuronal circuit analyzed. Gray indicates the presynaptic neuron expressing ChR2. (F) Average amplitude of the light induced EPSCs or IPSCs. (G) Average amplitude of the light-induced hyperpolarization (IPSP) or depolarization (EPSP), when the depolarization was not sufficient to trigger an action potential.

FIG. 4 illustrates RO4 and ChR2 can be used to regulate the frequency of spontaneous rhythmic activity in isolated embryonic chick spinal cords and living embryos. (A) Diagram of isolated chicken spinal cord preparation showing the position of the recording suction electrode; regions electroporated with either ChR2 or RO4 are shown in gray. (B) Electrical recording from motor nerve of ChR2 lumbar-electroporated embryo showing two control episodes in the absence of light (Upper) with an expanded time base trace of a single episode shown (Lower). Bursts of many motor axons firing synchronously and individual motor axons firing asynchronously are noted. (C) Plot of the intervals (in min) between bursting episodes from a lumbar electroporated ChR2 embryo subjected to a long interval of continuous light (circles) or 3-s pulses of light (triangles); filled symbols indicate episodes elicited in the presence of light, and open circles indicate episodes occurring in the absence of light. (D) Electrical recordings showing episodes (denoted by brackets) occurring during several minutes of continuous light (Upper) or elicited by a 3-s pulse of light at the position of the asterisk (Lower). (E) Comparison of unit activity preceding bursts that occurred spontaneously in a nonelectroporated embryo (Top) or were elicited by light when ChR2 was expressed selectively in the lumbar cord (Middle) or cervical cord (Bottom). Time of light exposure is indicated by dashed line. (F) Bar graph of the percent change in motor unit activity occurring in control embryo and one electroporated at cervical or lumbar level during a 3-s exposure to light. (G) The frequency of axial movements of stage 25-26 embryos in ovo, 3 days after ChR2 was electroporated into cervical cord segments, in the presence or absence of 475 nM light. (H) Plot of intervals between bursting episodes in embryos electroporated with RO4 at lumbar level when exposed to a long interval of continuous light (circles) or 3-s light pulses at different repetition rates (triangles); filled symbols indicate episodes occurring in the presence of light, open symbols indicate those that occurred in the absence of light. (I) Activation of RO4 by brief light pulses triggers bursting episodes. (Top) After a spontaneous episode (no. 1) a 2-s light pulse was able to trigger a premature bursting episode (no. 2); both are shown on expanded time bases in Middle and Bottom, respectively (see text for more detail). (J) Bar graph of change in motor unit activity in the period preceding the first burst of a spontaneous episode or one evoked by light activation of RO4. (K) Light activation of RO4 can synchronize the bursting behavior of spinal cord motoneurons. Right and left sides of a RO4 lumbar electroporated cord exhibit independent (asynchronous) rhythms when they are surgically separated at the midline (top pair of traces) However, the bursts triggered after the cessation of a light stimulus results in their synchronization (bottom pair of traces). LS3, lumbar segment 3; Sp.N., spinal nerve.

### DETAILED DESCRIPTION

The present invention relates to light-sensitive (or light-activated) transmembrane proteins and to systems and methods of using such transmembrane proteins for inducing cell apoptosis or death and/or modulating or controlling cell signaling. The light-sensitve transmembrane protein can be used to induce cell death and cell signaling in tissue and cells accessible to light. Examples of cell inducing and cell signaling applications include inducing cell death in neoplastic tissue that is accessible to light, such as tumors of the skin (e.g., melanoma) and tumors of epithelial cells (e.g., mouth and gut) of a subject being treated.

The system and methods of the present invention provide for the ability to control via specific wavelengths of light, including sun and room light, the activation or ion fluxes and G-protein signaling pathways. Activation of at least some of the transmembrane proteins can be mediated by the light-sensitive, retinal compound all-trans retinal. Therefore activation of channel/receptor of these light sensitive transmembrane proteins can occur only when two stimuli are provided, i.e., light and ligand.

The light-sensitive transmembrane can be expressed in cells that are accessible to light (e.g., sun light, light guides, ambient light). The cells can include, for example, skin cells and epithelial cells in the gut or mouth of a subject being treated. The subject being treated can include a mammal, such as a mouse, rat, and human. The skin cells or epithelial cells can comprise neoplastic or tumor cells in which it is desired to induce cell apoptosis.

Therapeutic uses of the light-sensitive transmembrane protein can include, for example, inducing cell death in tumors of the skin and tumors of epithelial cells (e.g., mouth and gut), modulating signaling in skin diseases, such as neurodermitis, and modulating cells signaling for cosmetic reasons.

The light-senstive transmembrane proteins can change ion fluxes over the cell membrane and/or change the polarization (e.g., hyperpolarize or depolarize) of the cell. Changes in ion fluxes over the cell membrane are associated with induced cell death (e.g., apoptosis). (See Franco et al., 2006, J. Membrane Biology, 209, 43-58, which is herein incorporated by reference in its entirety). Several ion conductances, for example, non-selective cation channels can induce apoptosis. Examples of light-sensitive transmembrane proteins that can activate cation channels include channel rhodoposins, such as ChR1, ChR2, and ChR3 (e.g., channel rhodoposin from Chlamydomonas reinhardtii). These light-sensitive transmembrane proteins when expressed in neoplastic cells or tumor cells of a subject being treated induce neoplastic or tumor cell death upon exposure to light.

By way of example, ChR2 a light activatable non-selective cation channel, which can be persistently opened during application of light, was expressed in HEK-293 cells. Exposure of the transfected cell to light induced ChR2 currents in the cells which in turn induced apoptosis and cell death.

Other light-sensitive transmembrane proteins that can be expressed in cells to induce cell death or signaling include light activated ion transporters, such as bacterio rhodopsin, vertebrate and invertebrate rhodopsins, and light activated adenylate cyclase (PAC).

In an aspect of the invention, the light-sensitive transmembrane proteins can be expressed in the cells using gene therapy. In an aspect of the invention, the gene therapy can use a vector including a nucleotide encoding the light-sensitive transmembrane protein. A “vector” (sometimes referred to as gene delivery or gene transfer “vehicle”) refers to a macromolecule or complex of molecules comprising a polynucleotide to be delivered to the cell. The polynucleotide to be delivered may comprise a coding sequence of interest in gene therapy. Vectors include, for example, viral vectors (such as adenoviruses (‘Ad’), adeno-associated viruses (AAV), and retroviruses), liposomes and other lipid-containing complexes, and other macromolecular complexes capable of mediating delivery of a polynucleotide to a target cell.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

Vectors for use in the present invention include viral vectors, lipid based vectors and other non-viral vectors that are capable of delivering a nucleotide according to the present invention to the target cells. The vector can be a targeted vector, especially a targeted vector that preferentially binds to neoplastic cells, such as cancer cells or tumor cells. Viral vectors for use in the invention can include those that exhibit low toxicity to a target cell and induce production of therapeutically useful quantities of the light-sensitive transmembrane protein in a cell specific manner.

Examples of viral vectors are those derived from adenovirus (Ad) or adeno-associated virus (AAV). Both human and non-human viral vectors can be used and the recombinant viral vector can be replication-defective in humans. Where the vector is an adenovirus, the vector can comprise a polynucleotide having a promoter operably linked to a gene encoding the light-sensitive transmembrane protein and is replication-defective in humans.

Other viral vectors that can be use in accordance with the present invention include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, might also be used in the invention. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and an the light-sensitive transmembrane protein nucleic acid. In methods of delivery to neoplastic cells, it may also encode a ligand to a tissue specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells. They are also highly efficient at transducing human epithelial cells.

Lentiviral vectors for use in the invention may be derived from human and non-human (including SIV) lentiviruses. Examples of lentiviral vectors include nucleic acid sequences required for vector propagation as well as a tissue-specific promoter operably linked to a light-sensititive transmembrane protein gene. These former may include the viral LTRs, a primer binding site, a polypurine tract, att sites, and an encapsidation site.

A lentiviral vector may be packaged into any suitable lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN), might also be used in the invention. Use of alphaviruses is described in Lundstrom, K., Intervirology 43:247-257, 2000 and Perri et al., Journal of Virology 74:9802-9807, 2000.

Recombinant, replication-defective alphavirus vectors are advantageous because they are capable of high-level heterologous (therapeutic) gene expression, and can infect a wide target cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell.

In many of the viral vectors compatible with methods of the invention, more than one promoter can be included in the vector to allow more than one heterologous gene to be expressed by the vector. Further, the vector can comprise a sequence which encodes a signal peptide or other moiety which facilitates expression of the light-sensitive transmembrane protein from the target cell.

To combine advantageous properties of two viral vector systems, hybrid viral vectors may be used to deliver a nucleic acid encoding a light-sensitive transmembrane protein to a target tissue. Standard techniques for the construction of hybrid vectors are well-known to those skilled in the art. Such techniques can be found, for example, in Sambrook, et al., In Molecular Cloning: A laboratory manual. Cold Spring Harbor, N.Y. or any number of laboratory manuals that discuss recombinant DNA technology. Double-stranded AAV genomes in adenoviral capsids containing a combination of AAV and adenoviral ITRs may be used to transduce cells. In another variation, an AAV vector may be placed into a “gutless”, “helper-dependent” or “high-capacity” adenoviral vector. Adenovirus/AAV hybrid vectors are discussed in Lieber et al., J. Virol. 73:9314-9324, 1999. Retrovirus/adenovirus hybrid vectors are discussed in Zheng et al., Nature Biotechnol. 18:176-186, 2000.

Retroviral genomes contained within an adenovirus may integrate within the target cell genome and effect stable gene expression.

Other nucleotide sequence elements, which facilitate expression of the light-sensitive transmembrane protein gene and cloning of the vector are further contemplated. For example, the presence of enhancers upstream of the promoter or terminators downstream of the coding region, for example, can facilitate expression.

In accordance with another aspect of the present invention, a tissue-specific promoter, can be fused to a light-sensitive transmembrane protein gene. By fusing such tissue specific promoter within the adenoviral construct, transgene expression is limited to a particular tissue. The efficacy of gene expression and degree of specificity provided by tissue specific promoters can be determined, using the recombinant adenoviral system of the present invention. Tumor specific promoters and vectors are known and disclosed in Lilihammer et al. 2005, Cancer Gene Ther. Nov. 12, 2004(11): 864-72, which is herein incorporated by reference in its entirety.

In addition to viral vector-based methods, non-viral methods may also be used to introduce a nucleic acid encoding a light-sensitive transmembrane protein into a target cell. A review of non-viral methods of gene delivery is provided in Nishikawa and Huang, Human Gene Ther. 12:861-870, 2001. An example of a non-viral gene delivery method according to the invention employs plasmid DNA to introduce a nucleic acid encoding a light-sensitive transmembrane protein into a cell. Plasmid-based gene delivery methods are generally known in the art.

Synthetic gene transfer molecules can be designed to form multimolecular aggregates with plasmid DNA. These aggregates can be designed to bind to a target cell. Cationic amphiphiles, including lipopolyamines and cationic lipids, may be used to provide receptor-independent nucleic acid transfer into target cells (e.g., neoplastic cells). In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Methods that involve both viral and non-viral based components may be used according to the invention. For example, an Epstein Barr virus (EBV)-based plasmid for therapeutic gene delivery is described in Cui et al., Gene Therapy 8:1508-1513, 2001. Additionally, a method involving a DNA/ligand/polycationic adjunct coupled to an adenovirus is described in Curiel, D. T., Nat. Immun. 13:141-164, 1994.

Additionally, the nucleic acid encoding the light-sensitive transmembrane protein can be introduced into the target cell by transfecting the target cells using electroporation techniques. Electroporation techniques are well known and can be used to facilitate transfection of cells using plasmid DNA.

Vectors that encode the expression of the light-sensitive transmembrane protein can be delivered to the target cell in the form of an injectable preparation containing pharmaceutically acceptable carrier, such as saline, as necessary. Other pharmaceutical carriers, formulations and dosages can also be used in accordance with the present invention.

Where the target cell comprises a tumor cell being treated, the vector can be delivered by direct injection at an amount sufficient for the light-sensitive transmembrane protein to be expressed to a degree, which allows for highly effective therapy. By injecting the vector directly into or about the periphery of the tumor, it is possible to target the vector transfection rather effectively, and to minimize loss of the recombinant vectors. This type of injection enables local transfection of a desired number of cells, especially within the tumor, thereby maximizing therapeutic efficacy of gene transfer, and minimizing the possibility of an inflammatory response to viral proteins. Other methods of administering the vector to the target cells can be used and will depend on the specific vector employed.

The light-sensitive transmembrane protein can be expressed for any suitable length of time within the target cell, including transient expression and stable, long-term expression. In one aspect of the invention, the nucleic acid encoding the light-sensitive transmembrane protein will be expressed in therapeutic amounts for a defined length of time effective to induce apoptosis of the transfected cells.

A therapeutic amount is an amount, which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. Specific dosages of proteins and nucleic acids can be determined readily determined by one skilled in the art using the experimental methods described below.

In an aspect of the invention, the system and methods of the present invention can be combined with a luciferase system. Co-expression of luciferase and the light-sensitive transmembrane protein in accordance with the invention in a neoplastic cell allows for internal activation (i.e., within the subject) of ion channel. This is important for performing experiments in living animals (e.g., humans) since the system can be activated by intake or infusion of luciferin in temporal manner.

Accordingly, one aspect of the present invention relates to a method of modulating cell signaling in neoplastic cells. In the method, light-sensitive transmembrane proteins are expressed from the neoplastic cells. The light sensitive transmembrane proteins modulate polarization of the cell upon exposure to a wavelength of light. The neoplastic cells expressing the light sensitive transmembrane proteins are exposed to the wavelength of light.

In an aspect of the invention, the modulation of cell signaling and/or the modulation of polarization of cell can induce cell apoptosis or death. The light-sensitive transmembrane protein can modulate ion transport or fluxes across the cell membrane upon exposure to light.

In another aspect of the invention, the neoplastic cells can comprise at least one of a tumor of skin or a tumor of epithelial cells. The light-sensitive transmembrane protein can comprise at least one of channelrhodopsin, bacteriorhodopsin, vertebrate rhodopsin, invertebrate rhodopsin, or light activatable adenylate cyclase. The channelrhodopsin can include at least one of ChR1, ChR2, or ChR3.

The present invention also relates to a method of inducing cell death in neoplastic cells. In the method, light-sensitive transmembrane proteins are expressed from the neoplastic cells. The light sensitive transmembrane proteins modulate polarization of the cell upon exposure to a wavelength of light. The neoplastic cells expressing the light sensitive transmembrane proteins are exposed to the wavelength of light. The light-sensitive transmembrane protein can comprise at least one of channelrhodopsin, bacteriorhodopsin, vertebrate rhodopsin, invertebrate rhodopsin, or light activatable adenylate cyclase. The channelrhodopsin can include at least one of ChR1, ChR2, or ChR3.

The present invention further relates to a neoplastic cell comprising a light-sensitive transmembrane protein. The light sensitive transmembrane proteins modulates polarization of the cell upon exposure to a wavelength of light. The light-sensitive transmembrane protein can comprise at least one of channelrhodopsin, bacteriorhodopsin, vertebrate rhodopsin, invertebrate rhodopsin, or light activatable adenylate cyclase. The channelrhodopsin can include at least one of ChR1, ChR2, or ChR3.

### Example

### Fast Noninvasive Activation and Inhibition of Neural and Network Activity by Vertebrate Rhodopsin and Green Algae Channel Rhodopsin

A major challenge in understanding the relationship between neural activity and development and between neuronal circuit activity and specific behaviors is to be able to control the activity of large populations of neurons or regions of individual nerve cells simultaneously. Recently, it was demonstrated that neuronal circuits can be manipulated by expressing mutated ion channels or G protein-coupled receptors (GPCRs). For example, the regional expression of a genetically modified K⁺ channel in Drosophila was able to reduce the excitability of targeted cells (i.e., muscle, neurons, photoreceptors). Silencing of cortical neurons was achieved by binding of the peptide allostatin to its exogenously expressed receptor. Activation and deactivation of neuronal firing could also be achieved when ligand-gated ion channels, such as the capsaicin receptor, menthol receptor, purinergic receptors, or light-controllable K⁺ channel blockers, were used to control firing in hippocampal neurons. However, the application of these techniques to control neuronal function especially in neural circuits and living animals is limited by their relatively slow time course, the complexity of the constructs to be expressed, or the requirement to apply and wash out ligands. To overcome these limitations, we developed molecular probes that could hyperpolarize or depolarize cells on a ms time scale and be used in intact vertebrate systems to examine behavior. To produce hyperpolarization of the somato-dendritic membrane or inhibition of synaptic transmitter release, the GPCR rat rhodopsin 4 (RO4), a member of the vertebrate rhodopsin family, that acts via the Gi/o pathway to regulate excitability by increasing somato-dendritic K⁺ and decreasing presynaptic Ca²⁺ conductances in neurons, was used. To depolarize the cell membrane, channel rhodopsin (ChR2) from the green algae Chlamydomonas reinhardtii, a cation selective channel directly gated by light, was expressed to produce a high Na⁺ conductance. The properties of these light-activated switches were extensively characterized and shown to be useful for modulating neuronal excitability and synaptic transmission in cultured hippocampal neurons. They were then introduced into the embryonic chick spinal cord and shown to be capable of controlling spontaneous rhythmic activity in isolated cords and living embryos.

### Materials and Methods

### Plasmid Constructs

For construction of ChR2(1-315)-GFP, RO4, and muscarinic acetylcholine receptor (mAChR)-M2 expression constructs and SinRep(nsP2S⁷²⁶)dSP-EGFP carrying RO4 and ChR2(1-315) virus constructs see Supporting Text, which is published as supporting information on the PNAS web site. Sindbis virus vector SinRep(nsP2S⁷²⁶) and helper DH-BB were kindly provided by P. Osten (Max Planck Institute for Medical Research, Heidelberg) and RO4 by A. Huber (University of Karlsruhe, Karlsruhe, Germany) (GenBank accession no. Z46957).

### Cell Culture

Culturing, maintaining, and transfection of human embryonic kidney (HEK) 293 cells (tsA201 cells) and low-density and autaptic hippocampal neurons were performed as described. To detect the distribution of RO4 and ChR2, neurons were transfected by using the calcium phosphate method.

### Viral Production and Infection

Sindbis pseudovirions were prepared according to Invitrogen's directions (Sindbis Expression System).

Viral titer was ≈1×10⁸ unit per ml stocked in −80° C. For neuronal infection, viral solution was added to cultured hippocampal neurons on coverslips in 24-well plates. Expression was detected after 10 h and reached maximal expression after 24 h.

### Immunocytochemistry and Image Acquisition

For transfection, immunostaining, and image acquisition of hippocampal neurons and spinal cord whole mounts see Supporting Text.

### Application of Retinal to RO4- or ChR2-Expressing Cells

Bath application of all-trans retinal [100 nM (Sigma)] 2 min before the experiment was sufficient for light activation of both proteins in all preparations tested, i.e., HEK293 cells, cultured hippocampal neurons, and isolated chicken spinal cord. Exogenous application of retinal compounds was not required for light-mediated activation of RO4 and ChR2 in chicken embryos in ovo.

### Electrophysiology and Data Analysis

All whole-cell patch-clamp recordings were performed. Recording solutions and conditions are given in Supporting Text. Illumination of patches was achieved with a TILL Photonics (Planegg, Germany) Polychrome II monochromator containing a 75-W xenon short arc lamp with an output of 250-690 nm and 475 nm was used to excite ChR2 or RO4. The light intensity was 1×10⁻⁶ W measured by power meter (Coherent, Santa Clara, Calif.), and the light source was controlled by the EPC9. Light and perfusion traces were programmed in PULSE software.

### Spinal Cord Preparation and Measurements.

In ovo electroporation, imaging of motor axons, recording of spontaneous bursting episodes in isolated spinal cord preparations, and the quantification of unit activity were. Statistical significance throughout the experiments was tested with ANOVA by using IGOR software. Standard errors are given as mean±SEM.

### Results

### Vertebrate Rhodopsin Can Be Used to Inhibit Neuronal Excitability and Synaptic Transmission

Vertebrate rhodopsin couples to the G protein transducin, the subunit of which belongs to the Gi subfamily, thus raising the possibility that mammalian rhodopsins would couple to other Gi/o family members. In neurons, the pertussis toxin (PTX)-sensitive Gi/o pathway activates G protein inward rectifying potassium channels (GIRKs) and inhibits presynaptic voltage-gated Ca²⁺ channels. GIRK channels are predominantly expressed on dendrites where they can hyperpolarize neurons. Presynaptic Ca2⁺ channels control transmitter release and inhibiting them via Gi/o-coupled receptors inhibits Ca2⁺ influx and transmitter release.

To determine whether vertebrate rhodopsin could be used as a light-activated switch to reduce neuronal excitability postsynaptically and transmitter release presynaptically, RO4 was coexpressed with either GIRK channel subunits 1 and 2 or the P/Q-type Ca2⁺ channel consisting of the α₁2.1, β_{1b}, and α2δ subunits. The mAChR M2 (mAChR-M2) was also expressed to serve as a positive control for G protein modulation of GIRK and presynaptic Ca2⁺ channels via Gi/o-PTX-sensitive GPCRs, because it modulates both GIRK and P/Q-type Ca2⁺ channels in vivo and in heterologous expression systems. We first demonstrated in HEK cells that both of these channels were modulated by light activation of RO4 in a manner very similar to their modulation via mAChR-M2.

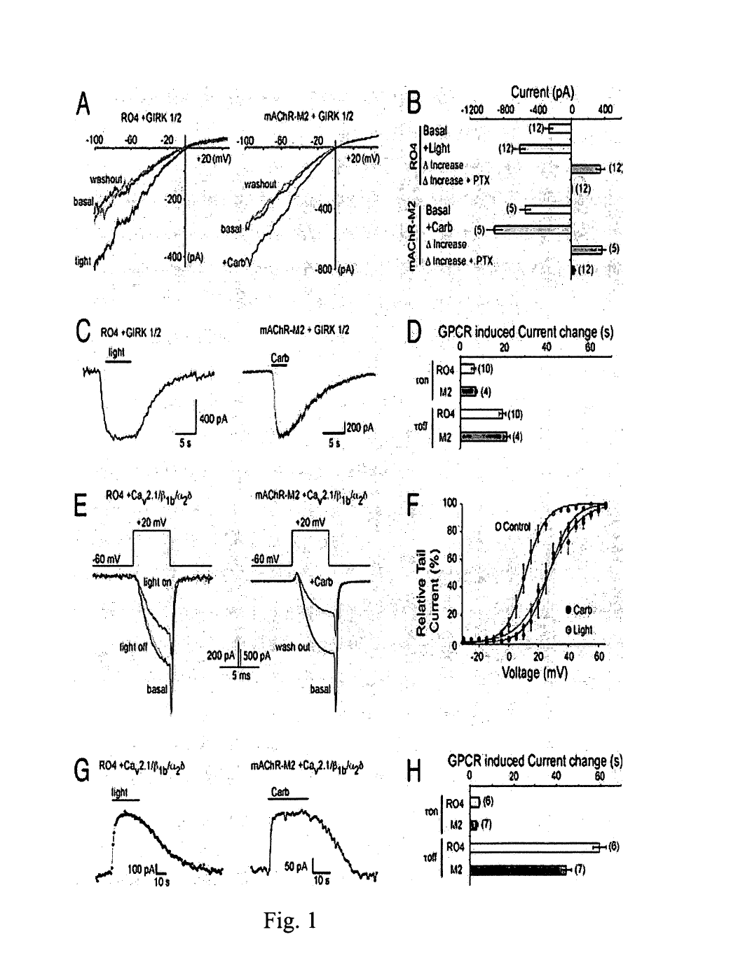

Activation of the GPCRs by either light or the AChR agonist carbachol (Carb) increased GIRK-mediated K+ currents by comparable amounts (FIGS. 1A and B) and with comparable activation and deactivation kinetics (FIGS. 1C and D). Importantly, light activation of RO4 was blocked by prior application of PTX, indicating that activation of GIRK channels by vertebrate rhodopsin is mediated via PTX-sensitive pathways (FIG. 1B). The amount of desensitization during long light or ligand exposure times was modest and comparable between the two [8.7±0.8% (n=4) for mAChR-M2 and 8.7±1.1% (n=4) for RO4], indicating that RO4 can be activated by light over long time periods. When RO4 and mAChR-M2 were coexpressed with the P/Q-type Ca²⁺ channel, light caused reversible inhibition of the Ca²⁺ currents (FIGS. 1E and G and FIG. 5, which is published as supporting information on the PNAS web site). Light or Carb caused a similar shift in the voltage dependence of activation to more depolarized potentials (FIG. 1F). In addition, the G protein inhibition caused by light was reversed by high positive prepulses applied shortly before a test pulse over a voltage range between −10 and −65 mV (data not shown) similar to the inhibition caused by Carb. Furthermore, lightmediated channel inhibition was inhibited by PTX. The time constants for onset of inhibition and reversal of inhibition were also comparable between RO4 and mAChR-M2 (τₒₙ=3-7 s, τ_{off}≈20-60 s, FIGS. 1G and 1H. Thus, vertebrate rhodopsin modulates GIRK and P/Q-type Ca²⁺ channels via PTX-sensitive pathways with similar efficacy and activation and deactivation kinetics as the mAChR.

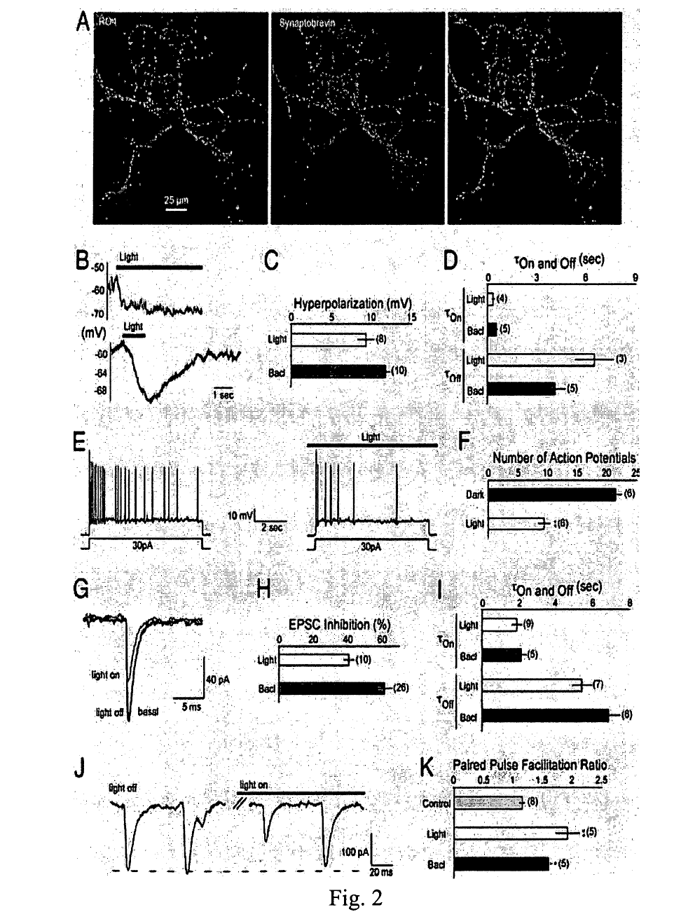

Because RO4 activates GIRKs, which control excitability postsynaptically, and inhibits Ca²⁺ channels of the Caᵥ2 family, which trigger transmitter release presynaptically, we next investigated in cultured hippocampal neurons whether light activation of RO4 could hyperpolarize neurons somato-dendritically to decrease their firing as well as inhibit presynaptic Ca²⁺ influx to modulate short-term synaptic plasticity such as paired-pulse facilitation. Exogenously expressed RO4 was localized somato-dendritically and transported to 70-80% of the synaptic sites where it colocalized with the presynaptic neuronal marker synaptobrevin II (FIG. 2A). Light activation of RO4 induced a 9-mV hyperpolarization within ms comparable to the hyperpolarization induced by activation of endogenous GABA_{B} receptors by 50 μM baclofen (FIGS. 2B and C). The hyperpolarization was stable during light application (measured up to 30 s) but was rapidly reversed when the light was switched off (FIGS. 2B and D). The time constants for hyperpolarization and repolarization were much faster than in HEK293 cells (compare FIGS. 2D and 1C) probably because of the effect of endogenous proteins, such as RGS proteins, which accelerate the GTPase activity of the G proteins. These observations are comparable to the described actions of Gi/o-coupled receptors on membrane changes in neurons (20). More importantly, the hyperpolarization induced by light was capable of reducing the number of action potentials produced during a depolarizing current pulse (FIGS. 2E and F).

Because RO4 appeared to be localized at synapses and inhibits P/Q-type Ca²⁺ channels in HEK293 cells, we investigated whether light activation of RO4 could be used to control presynaptic function. We analyzed facilitation properties before and after light application and compared these to the effect of activating the GABA_{B} receptor with baclofen (FIG. 2G-K). Light activation of RO4 reduced the excitatory postsynaptic current (EPSC) amplitude by 40% compared with 60% when the GABAB receptor was activated (FIGS. 2G and H), presumably because of a reduction in quantal content. The time constants for these effects were comparable for both receptors [τₒₙ=0.3-0.6 s,τ_{off}≈4-6 s (FIG. 2I)]. As would be expected if this reduction of EPSC amplitude was caused by a reduction in quantal content, paired-pulse facilitation for both receptor types was increased (FIGS. 2J and K). Taken together, these results show that light activation of RO4 can be used to control cell excitability via hyperpolarization of the somatodendritic membrane as well as presynaptically via reduction of transmitter release.

### Green Algae ChR2 Can Be Used to Precisely Drive

### Neuronal Firing on a Fast (ms) Time Scale

ChRs are microbial type rhodopsins with an intrinsic light-gated cation conductance. ChR1 from C. reinhardii is specific for protons, whereas ChR2 is a less selective cation channel with conductance for H⁺>>Na⁺>K⁺>Ca2⁺. Because the conductance of ChR2 is higher than that of ChR1 and the C terminally truncated version of ChR2 (1-315) is as active as the full-length protein, all experiments were carried out with the ChR2 (1-315) fragment fused to GFP at the C-terminal end of ChR2 (1-315) (7). To test whether the ChR2 can act to depolarize cells when activated by light, ChR2 (1-315) was first expressed and extensively characterized in HEK293 cells (FIG. 6, which is published as supporting information on the PNAS web site). Light activation of ChR2 was found to cause depolarizations of 10-25 mV within 10 ms, with repolarization occurring within 200 ms. Thus ChR2 should be capable of depolarizing neurons sufficiently to elicit action potentials.

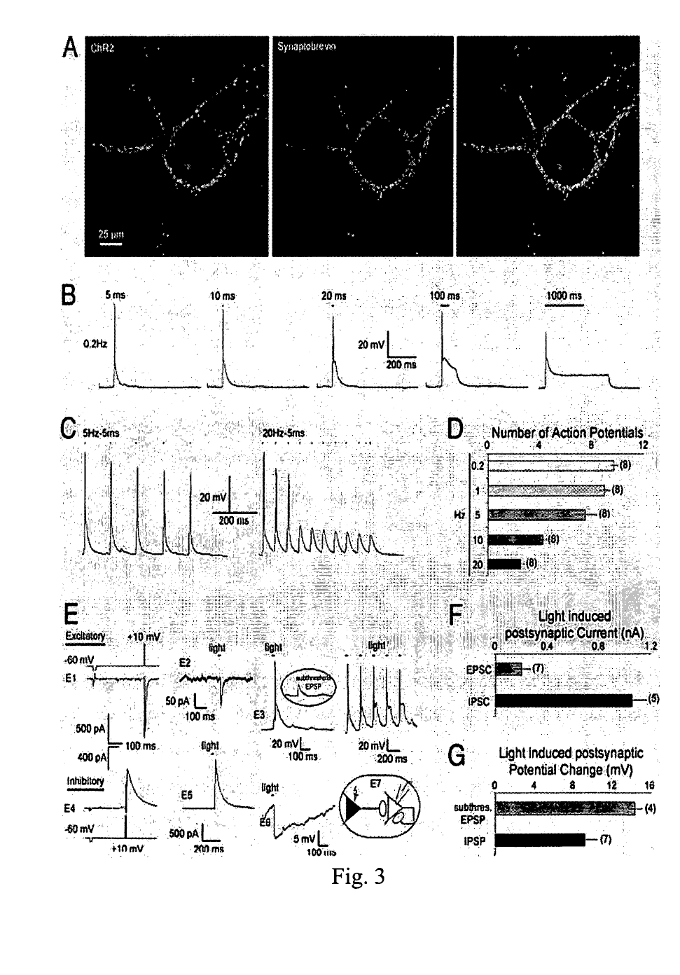

When exogenously expressed in hippocampal neurons, ChR2 appeared to localize both somato-dendritically and at 50-70% of the synaptic sites defined by synaptobrevin 2 immunostaining (FIG. 3A). A 5-ms light activation was sufficient to elicit action potentials in >90% of the experiments performed, whereas longer light exposure led to continuous subthreshold depolarization of the neurons (FIG. 3B). When stimulated at 5 Hz most stimuli elicited action potentials, but as the frequency of stimulation was increased, the proportion that triggered subthreshold EPSPs increased (FIGS. 3C and D). We next tested whether presynaptically expressed ChR2 was capable of triggering synaptic transmission on postsynaptic neurons. Pairs of hippocampal neurons were analyzed, in which a GFP-ChR2 expressing neuron synapsed with a ChR2-negative neuron that had formed autapses on its own soma (FIG. 3E, E₇ diagram). We found that inhibitory postsynaptic currents (IPSCs) as well as EPSCs could be successfully triggered by light activation of the presynaptic neuron (FIG. 3E). The light-activated currents were different in amplitude than the autaptic currents elicited by electrically stimulating the postsynaptic neuron (FIG. 3E), indicating that they are mediated through different neuronal contacts. In three of seven experiments light-activated postsynaptic EPSCs were sufficient to trigger somato-dendritic firing up to 20 Hz. In the remaining four experiments subthreshold EPSPs were observed (FIG. 3E, E₃). Light-induced postsynaptic IPSCs caused somatodendritic hyperpolarization (FIG. 3E, E₆). As expected the IPSC/EPSC amplitudes and degree of hyperpolarization or depolarization varied between analyzed neuronal pairs, as they would depend on the amount of synaptic contacts formed between the presynaptic and postsynaptic neuron (FIGS. 3F and G).

### Activation of RO4 and ChR2 Can Be Used to Control Spontaneous Activity in Isolated Intact Spinal Cords and Living Embryos

Our next goal was to show that these light-sensitive proteins could be used to control circuit behavior in whole animal preparations. Early embryonic chick spinal cords exhibit rhythmic episodes of spontaneous bursting activity, which are generated by recurrent excitatory connections between motoneurons and GABAergic and glycinergic intemeurons, all of which are excitatory at this stage of development. Recently, it has been shown that the normal pattern and frequency of this early spontaneous activity is required for appropriate motor axon path finding in the chick and for the development of cord circuits that enable appropriate flexor extensor and right-left phasing during locomotor-like activity in the mouse.

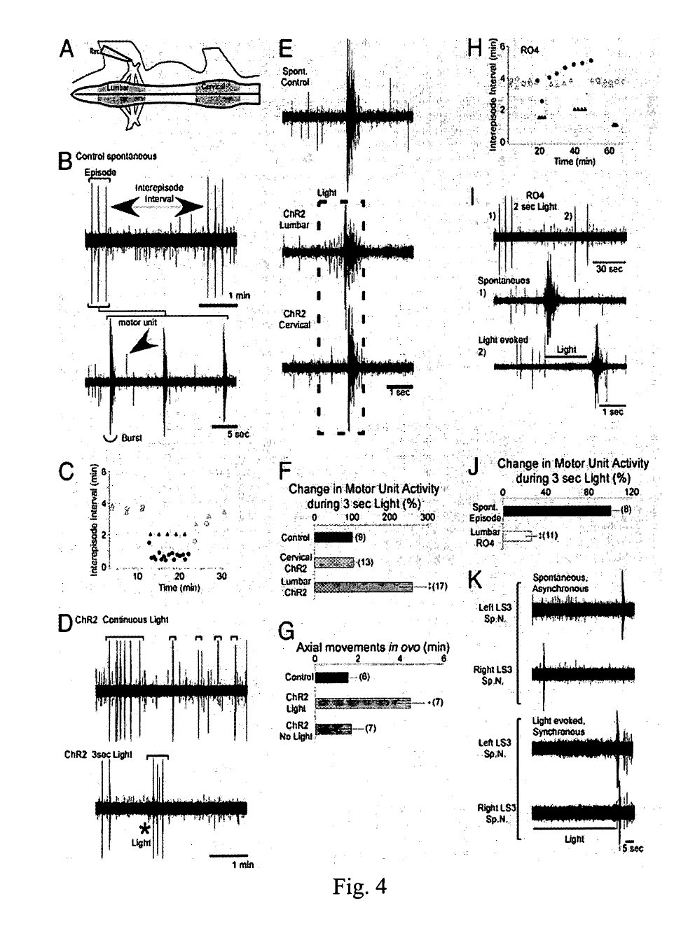

To assess whether such network activity, especially the frequency of spontaneous bursting episodes, could be controlled noninvasively by light, constructs for GFP-ChR2 or GFP-RO4 under the control of the CMV promoter were electroporated into the spinal cords of stage 16 (embryonic day 2-3) chick embryos in ovo. At stage 26 (embryonic day 4.5-5) isolated spinal cord-hind limb preparations were made, and the constructs were found to be expressed in many neurons including motor and interneurons and could be expressed selectively in lumbar or cervical cord by varying the electroporation protocol. Suction electrode recordings from lumbar motor nerves (FIGS. 4A and B) revealed that as in control embryos the electroporated embryos exhibited episodes consisting of several bursts every 4 min (FIGS. 4B and C). Thus, the electroporation protocol and expression of these constructs over several days did not appear to have any adverse effects on the development of the cord circuits responsible to generating this activity. The asynchronous firing of individual motoneurons between bursts and between episodes could also be detected (FIG. 4B, arrow). When exposed to continuous light (FIG. 4C, ) the interepisode intervals in this cord, electroporated at the lumbar level with ChR2, were shortened to <1 min. They were, however, less rhythmic than control spontaneous episodes and consisted of single bursts (FIG. 4D Upper). In contrast, the application of a 3-s light pulse was able to elicit a normal three-burst episode shortly after a spontaneous episode (FIG. 4D Lower), and such pulses when repeated could drive episodes at precise frequencies, in the example shown (FIG. 4C, ▴) at 2-min intervals. The expanded time base traces (FIG. 4E) show that light first elicited an increase in lumbar motor unit firing that subsequently resulted in a burst very similar to spontaneous episodes in nonelectroporated embryos. However, when expression of ChR2 was restricted to the cervical cord, lumbar motor nerve recordings revealed that it was also possible to drive episodes in the lumbar cord by light without a previous increase in lumbar unit activity, by generating episodes that propagated from the cervical level (FIGS. 4E and F). Thus light, as has been previously shown for electrical stimulation, can be used to elicit episodes either by activation of local lumbar interneurons and motoneurons or activation of neurons many cord segments distant.

To assess whether light could be used to drive rhythmic activity in intact embryos in ovo, axial movements, which are precisely correlated with electrically recorded episodes of activity, were videotaped under red light that did not activate the cervically electroporated ChR2. When several light pulses of the wavelength necessary to activate ChR2 were given through a window in the shell, each elicited a clear movement episode. Furthermore, a significant increase in the frequency of axial movements could be maintained by continuous application of light over several minutes (FIG. 4G). These observations indicate that the light switches can act in intact animal preparations without application of all-trans retinal (see Discussion) and that the light used is able to penetrate through the amnion and layers of tissue to activate the spinal cord neurons.

Because light activation of RO4 hyperpolarized hippocampal neurons, we next explored whether it could be used to suppress spontaneous bursting activity. During continuous light, the interval between spontaneous episodes increased only modestly in cords with lumbar expression of RO4 (FIG. 4H, ). This finding was not entirely unexpected because regions of cord not electroporated with RO4 would still be able to depolarize and contribute to the excitation required to elicit a bursting episode (see ref. 23) for details of episode generation). Surprisingly, however, a 2-s pulse of light actually elicited a premature episode (4I, 2) 1 min after a spontaneous episode (FIG. 4I, 1). Yet when 1-, 1.5-, or 2-s pulses of light were given, lumbar motor unit activity was suppressed during the light and the episode was triggered only when the light was switched off (FIG. 4I, 2). During the light exposure asynchronous firing of motoneurons was also suppressed (FIG. 4I Bottom and J). Thus, while the activation of RO4 in intact cord circuits could affect excitability by the activation of other G protein-coupled pathways, for example, by activating glycine receptors that are excitatory at this stage, our results suggest that in the embroynic day 5 chick cord hyperpolarization of the transfected neurons predominates. We propose that such hyperpolarization of cells within the circuit, possibly by relieving the inactivation of voltage-gated Na+ channels, enhances the probability that these cells will fire together, when the light is extinguished and thus provides another means for synchronizing bursting episodes within the circuit. Thus light activation of RO4 could precisely drive episodes at 1-, 1.5-, or 2-s intervals (FIG. 4H, ▴). In addition, when the connections between the right and left sides of the cord are surgically severed, the episodes on the two sides occur asynchronously, but can be synchronized by light activation of RO4 (FIG. 4K).

### Discussion

This study has shown that vertebrate rhodopsin RO4 and green algae ChR2 can be used to control neuronal function when activated by light. RO4 acted postsynaptically to hyperpolarize neurons and inhibit action potential firing and presynaptically to reduce transmitter release. We also demonstrated that ChR2 could function somato-dendritically to depolarize neurons and cause action potential firing. Whether it is transported to the presynaptic terminal where currents generated by it could modulate transmission remains to be determined. However, the transport of RO4 to presynaptic sites, where it was capable of modulating presynaptic function (transmitter release and paired-pulse facilitation), suggest that it will be a useful tool for studying G protein-mediated effects at the vertebrate presynaptic terminal in the ms time range and will provide a means for precise temporal activation and deactivation of presynaptic G proteins. Such precise activation is not possible with activating GPCRs with ligands, because washout, transport, or degradation of the ligands is slow. It is likely that ms activation of presynaptic terminal G proteins will lead to insights into the presynaptic function of G proteins, and in particular for events involved in short-term synaptic plasticity and modulation of transmitter release.

ChR2, which appears to be the protein of choice for increasing excitability and firing of neurons, was also very recently characterized. We observed that light stimulation frequencies >5 Hz led to a decrease in the success rate of action potential firing, probably because of the use-dependent decrease in ChR2 currents combined with a frequency-dependent increase in Na+ channel inactivation. The 5-Hz stimulation protocol, which we found resulted in a high success rate in eliciting trains of action potentials, is in agreement with the 200-ms time it takes to recover from the ChR2-induced depolarization. Thus, the extent to which a neuron will be able to precisely follow the frequency of light pulses will probably depend on the membrane properties of the different classes of neurons.

A potential concern related to the use of light-activated switches is the extent to which the light will penetrate tissues. However, we demonstrated here that the applied light was sufficient to activate both isolated spinal cords and intact embryonic day 5-6 chick embryos inside the egg, where light was applied through a window in the shell. Furthermore, the fact that light stimuli could be applied to the chick cords over many hours without altering the pattern or frequency of the spontaneous rhythmic activity in the absence of light suggests that the light has not damaged the complex cord circuits required for generating this activity. Taken together, our experiments thus demonstrate that neuronal circuits within intact embryos can be controlled by a noninvasive technique without the need for any chemical compounds.

Thus, the light switches we have developed should provide important tools for characterizing cell and network function in living animals or tissue. Placing these switches under the control of specific promoters will enable one to control the activity of specific subsets of neurons and thus determine their role in complex behaviors, as, for example, defining the roles of subclasses of interneurons and motoneurons in locomotion. Besides their utility for basic characterization of neuronal circuit function and behavior, these proteins will provide additional tools for developing externally, light-controlled molecular machines to circumvent disease or trauma-induced alterations in nervous system excitability, such as after spinal cord injuries, heart arrhythmia, and Parkinson's disease.

1. A method of modulating cell signaling in neoplastic cells:
expressing light-sensitive transmembrane proteins in the neoplastic cells, the light sensitive transmembrane proteins modulating polarization of the cell upon exposure to a wavelength of light, and
exposing the neoplastic cells expressing the light sensitive transmembrane proteins to the wavelength of light.

2. The method of claim 1, the modulation of cell signaling inducing cell death in the neoplastic cell.

3. The method of claim 1, the neoplastic cells comprising at least one of a tumor of skin or a tumor of epithelial cells.

4. The method of claim 1, the modulation of polarization of cell inducing cell death.

5. The method of claim 1, the light-sensitive transmembrane protein modulating ion fluxes across the cell membrane upon exposure to light.

6. The method of claim 1, the light-sensitive transmembrane protein comprising at least one of channelrhodopsin, bacteriorhodopsin, vertebrate rhodopsin, invertebrate rhodopsin, or light activatable adenylate cyclase.

7. The method of claim 1, the light-sensitive transmembrane comprising channelrhodopsin 2.

8. The method of claim 7, the channelrhodopsin being expressed in tumor cells of the skin.

9. A method of inducing cell death in neoplastic cells,
expressing light-sensitive transmembrane proteins in the neoplastic cells, the light sensitive transmembrane proteins modulating polarization of the cell upon exposure to a wavelength of light; and
exposing the neoplastic cells expressing the light sensitive transmembrane proteins to the wavelength of light.

10. The method of claim 9, the neoplastic cells comprising at least one of a tumor of skin or a tumor of epithelial cells.

11. The method of claim 9, the modulation of polarization of cell inducing cell death.

12. The method of claim 9, the light-sensitive transmembrane protein modulating ion fluxes across the cell membrane upon exposure to light.

13. The method of claim 9, the light-sensitive transmembrane protein comprising at least one of channelrhodopsin, bacteriorhodopsin, vertebrate rhodopsin, invertebrate rhodopsin, or light activatable adenylate cyclase.

14. The method of claim 9, the light-sensitive transmembrane comprising channelrhodopsin 2.

15. The method of claim 14, the channelrhodopsin being expressed in tumor cells of the skin.

16-19. (canceled)
